(19) United States
(12) Patent Application Publication
(10) Pub. No.: US 2007/0060549 A1
(43) Pub. Date: Mar. 15, 2007

(54) COMBINATION THERAPIES EMPLOYING ACE INHIBITORS AND USES THEREOF FOR THE TREATMENT OF DIABETIC DISORDERS

(76) Inventor: Albert D. Friesen, Winnipeg (CA)

Correspondence Address: MERCHANT & GOULD PC, P.O. BOX 2903, MINNEAPOLIS, MN, 55402-0903, US

(21) Appl. No.: 11/424,069

(22) Filed: Jun. 14, 2006

Related U.S. Application Data
(63) Continuation-in-part of application No. 11/202,272, filed on Aug. 10, 2005.
(60) Provisional application No. 60/599,866, filed on Aug. 10, 2004.

(51) Int. Cl. A61K 31/675; A61K 31/401

(52) U.S. Cl. 514/89; 514/423

(57) ABSTRACT

The present invention includes use of an angiotensin-converting enzyme (ACE) inhibitor in combination with a vitamin B6 related compound for the treatment of diabetes and diabetic related disorders and in particular the treatment of diabetic hypertension.

### CROSS REFERENCE TO RELATED APPLICATIONS

This application is a continuation-in-part of U.S. application Ser. No. 11/202,272, filed Aug. 10, 2005, which claims the benefit under 35 U.S.C. §119(e) of U.S. Provisional Patent Application No. 60/599,866, filed Aug. 10, 2004, the entire disclosures of which are hereby incorporated by reference.

### FIELD OF INVENTION

The present invention relates to combination therapies employing angiotensin converting enzyme (ACE) inhibitors and uses thereof, and in particular the use of such combination therapies for the treatment of diabetic disorders.

### BACKGROUND

Hypertension is an extremely common co-morbid condition in diabetics, affecting up to 11 million patients. Hypertension substantially increases the risk of both macrovascular and microvascular complications including stroke, coronary artery disease, peripheral vascular disease, retinopathy, nephropathy and possibly neuropathy.

In recent years, clinical trials have indicated that aggressive treatment of hypertension may reduce diabetic complications. In the epidemiological UK Prospective Diabetes Study (UKPDS), each 10 mmHg decrease in mean systolic blood pressure was associated with reductions in risk of 12% for any complication related to diabetes, 15% for deaths related to diabetes, 11% for myocardial infarction and 13% for microvascular complications. Currently the consensus guidelines recommend a blood pressure target of <130/80 mmHg in diabetic patients with hypertension, even though they recognize many people will require three or more drugs to reach this goal.

Hypertension is twice as common in people with diabetes as compared to the rest of the population. Recent clinical studies have shown that despite substantial clinical research and refinements to existing pharmacological therapy, the ability to control hypertension remains at the same level as in the 1980s. Accordingly, there is a need for more effective anti-hypertesive therapies, and especially therapies useful for the treatment of diabetic hypertension.

### SUMMARY OF INVENTION

A first aspect of the present invention provides a method of treating or inhibiting hypertension in a diabetic patient in need thereof, comprising administering a therapeutically effective dose of an ACE inhibitor and a vitamin B6 related compound.

A second aspect of the present invention provides a method of improving kidney function in a diabetic patient in need thereof comprising administering a therapeutically effective amount of ACE inhibitor and a vitamin B6 related compound.

A third aspect of the present invention provides a method of treating or inhibiting nephropathy in a diabetic patient in need thereof comprising administering a therapeutically effective amount of an ACE inhibitor and a vitamin B6 related compound.

A fourth aspect of the present invention provides a method of improving metabolic function in a diabetic patient in need thereof, comprising administering a therapeutically effective dose of an ACE inhibitor and a vitamin B6 related compound.

In an embodiment, the metabolic function to be improved includes: increased insulin sensitivity, increased glycemic control, decreased insulinemia, decreased hyperglycemia, decreased hyperlipidemia or a combination thereof.

A fifth aspect of the present invention provides a method of improving endothelial function in a diabetic patient in need thereof, comprising administering a therapeutically effective dose of an ACE inhibitor and a vitamin B6 related compound.

A sixth aspect of the present invention provides a method of improving vascular function in a diabetic patient in need thereof, comprising administering a therapeutically effective dose of an ACE inhibitor and a vitamin B6 related compound.

In an embodiment of the invention, a vitamin B6 related compound can include pyridoxal, pyridoxal-5′-phosphate, pyridoxamine, a 3-acylated analogue of pyridoxal, a 3-acylated analogue of pyridoxal-4,5-aminal, a pyridoxine phosphate analogue, or a mixture thereof.

In a further embodiment of the invention, an ACE inhibitor can include benazepril; captopril; cilazapril; enalapril; enalaprilat; fosinopril; lisinopril; moexipril; perindopril; quinapril; ramipril; trandolapril; or a mixture thereof.

In yet a further embodiment of the invention, the ACE inhibitor is lisinopril and the vitamin B6 related compound is pyridoxal-5′-phosphate.

### BRIEF DESCRIPTION OF THE FIGURES

FIG. 1 shows the change in mean daytime ambulatory systolic blood pressure (MDASBP) from baseline (ΔBP) after 8 weeks, measured in mmHg. Patients were treated with lisinopril alone (L), 100/20 (pyridoxal-5′-phosphate (P5P)/L) mg/dose, 300/20 (P5P/L) mg/dose, or 1000/20 (P5P/L) mg/dose.

FIG. 2 shows the change in mean daytime ambulatory diastolic blood pressure (MDADBP) from baseline (ΔBP) after 8 weeks, measured in mmHg. Patients were treated with lisinopril plus placebo (L), 100/20 (P5P/L) mg/dose, 300/20 (P5P/L) mg/dose, or 1000/20 (P5P/L) mg/dose.

FIG. 3 shows the placebo-corrected reduction in fasting serum glucose (FSG) from baseline (ΔFSG), measured in mmol/L. Patients were treated with 100/20 (P5P/L) mg/dose, 300/20 (P5P/L) mg/dose, or 1000/20 (P5P/L) mg/dose for 8 weeks and the same dosages of P5P without lisinopril for 8 weeks.

FIG. 4 shows the reduction in fasting serum glucose (FSG) from baseline (ΔFSG) in patients with a FSG of 10 mmol/L or greater at baseline. Patients were treated with lisinopril plus placebo (L), 100/20 (P5P/L) mg/dose, 300/20 (P5P/L) mg/dose, or 1000/20 (P5P/L) mg/dose for 8 weeks and the same dosages of P5P or placebo without lisinopril for 8 weeks.

FIG. 5 shows the placebo-corrected reduction (% change) in glycated hemoglobin (HbA1c) from baseline (ΔHbA1c) in patients with a HbA1c of 8% or greater at baseline. Patients were treated with 100/20 (P5P/L) mg/dose, 300/20 (P5P/L) mg/dose, or 1000/20 (P5P/L) mg/dose for 8 weeks and the same dosages of P5P or placebo without lisinopril for 8 weeks.

FIG. 6 shows the reduction in triglyceride (TG) levels from baseline in patients with a triglyceride level of 1.7 mmol/L or greater at baseline. Baseline levels of triglycerides were established (▪) and then measured again after 16 weeks of therapy (□). Patients were treated with lisinopril plus placebo (L), 100/20 (P5P/L) mg/dose, 300/20 (P5P/L) mg/dose, or 1000/20 (P5P/L) mg/dose for 8 weeks and the same dosages of P5P or placebo without lisinopril for 8 weeks.

FIG. 7 shows the reduction in total cholesterol (TC) levels from baseline in patients with a total cholesterol level of 5.2 mmol/L or greater at baseline. Baseline levels of total cholesterol were established (▪) and then measured again after 16 weeks of therapy (□). Patients were treated with lisinopril plus placebo (L), 100/20 (P5P/L) mg/dose, 300/20 (P5P/L) mg/dose, or 1000/20 (P5P/L) mg/dose for 8 weeks and the same dosages of P5P or placebo without lisinopril for 8 weeks.

FIG. 8 shows the placebo-corrected reduction in low density lipoprotein (LDL) from baseline (ΔLDL), measured in mmol/L. Patients were treated with 100/20 (P5P/L) mg/dose, 300/20 (P5P/L) mg/dose, or 1000/20 (P5P/L) mg/dose for 8 weeks and the same dosages of P5P or placebo without lisinopril for 8 weeks.

FIG. 9 shows the reduction in low density lipoprotein cholesterol (LDL) levels from baseline in patients with a LDL level of greater than 2.6 mmol/L or greater at baseline. Baseline levels of LDL were established (▪) and then measured again after 16 weeks of therapy (□). Patients were treated with lisinopril plus placebo (L), 100/20 (P5P/L) mg/dose, 300/20 (P5P/L) mg/dose, or 1000/20 (P5P/L) mg/dose for 8 weeks and the same dosages of P5P or placebo without lisinopril for 8 weeks.

### DETAILED DESCRIPTION

Hypertension is a predictor of microvascular (e.g. renal and retinal) and cardiovascular (e.g. coronary, cerebrovascular, peripheral artery disease) complications of diabetes. Co-existence of hypertension and hyperglycemia dramatically and synergistically increases the risk of these complications. Active blood pressure reduction to <130/80 mmHg reduces the risk of diabetic complications. Recent data from the United Kingdom Prospective Diabetes Study underscores the importance of rigorous blood pressure control which may require several antihypertensive medications. Results from a number of clinical trials indicate that combination therapy should include an angiotensin converting enzyme (ACE) inhibitor for maximal benefits in protecting against cardiovascular disease (CVD) as well as renal disease.

As used herein, the term “vitamin B6 related compound” means any vitamin B6 related precursor, metabolite, derivative, or analogue. In a preferred embodiment, the vitamin B6 related compound used to practice the invention is pyridoxal-5′-phosphate (P5P). Other vitamin B6 related compounds which can also be used to practice the invention, include the 3-acylated analogues of pyridoxal, 3-acylated analogues of pyridoxal-4, 5-aminal, and pyridoxine phosphonate analogues described in U.S. Pat. No, 6,585,414 and US Patent Publication No. 2003/0114424, both of which are incorporated herein by reference.

By an “effective amount” or a “therapeutically effective amount” of a drug or pharmacologically active agent is meant a nontoxic but sufficient amount of the drug or agent to provide the desired effect. In the combination therapy of the present invention, an “effective amount” of one component of the combination is the amount of that compound that is effective to provide the desired effect when used in combination with the other components of the combination. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

The present inventors have previously reported the usefulness of vitamin B6 related compounds, and in particular pyridoxal-5′-phosphate (P5P), for the treatment of cardiovascular disorders, including essential hypertension (see U.S. Pat. Nos. 6,043,259 and 6,677,356). The inventors have now determined that vitamin B6 related compounds are particularly effective for treating or inhibiting diabetic hypertension and surprisingly, for treating or inhibiting a variety of diabetic disorders.

Vitamin B6 related compounds positively influence insulin sensitivity, glycemic control, and lipid levels in individuals with either type 1 (insulin dependent) or type 2 (insulin independent) diabetes. The present invention is further based on the discovery that the positive effects resulting from the administration of vitamin B6 related compounds to diabetics are enhanced when the vitamin B6 related compound is co-administered with an ACE inhibitor. Combination therapy comprising a vitamin B6 related compound and an ACE inhibitor is found to significantly improve metabolic, endothelial, and vascular function in individuals with either type 1 or type 2 diabetes, and pre-diabetic conditions. The antihypertensive effects of vitamin B6 related compounds and of ACE inhibitors were also found to be enhanced when the two classes of agents were co-administered to diabetic individuals.

Diabetics with hypertension are generally insulin resistant, glucose tolerant, hyperinsulinemic, dyslipidemic, and have endothelial dysfunction. It appears that insulin resistance and/or compensatory hyperinsulinemia play a role in blood pressure regulation and may play a role in predisposing individuals to develop high blood pressure (Reaven, G., J. Clin. Hypertens. 5(4):269-274, 2003).

While the present invention is not limited to any particular theory, vitamin B6 related compounds appear to positively influence metabolic, endothelial, and vascular function in diabetic individuals. The inventors have discovered that vitamin B6 related compounds, and in particular P5P, appear to increase insulin sensitivity and improve glycemic control. Furthermore, the beneficial modulation of metabolic function is enhanced when the vitamin B6 related compound is coadministered with an ACE inhibitor. The present inventors are the first to report the use of a vitamin B6 related compound, and in particular, the use of pyridoxal-5′-phosphate (P5P), alone or in combination with an ACE inhibitor, for the treatment of diabetes and diabetes related complications.

Diabetic patients treated with P5P were found to have improved metabolic function. It would appear that P5P improves insulin sensitivity in diabetics, and in particular type 2 diabetics. Glycated hemoglobin (HbA1c) is a biomarker used to measure blood glucose control. Glucose is carried in the blood stream and becomes attached to the hemoglobin molecule. As a result of this attachment, changes occur which can be measured to estimate the average glucose level for the life of the hemoglobin molecule. HbA1c measurement is the primary measure of glucose control used by the FDA to determine the efficacy of drug candidates in diabetics. The present inventors have discovered that diabetics treated with P5P alone had reduced HbA1c levels as compared to those individuals treated with a placebo. Additionally, the P5P individuals were found to not only have improved insulin sensitivity and glucose control, but also improved lipid profile (increased HDL levels, decreased LDL and triglyceride levels), improved endothelium function as evidenced by decreased levels of the cell adhesion markers and improved vascular function including improved blood pressure regulation. It is now shown that blood pressure regulation is further enhanced when a diabetic individual is administered P5P in combination with an ACE inhibitor. While the mechanism by which vitamin B6 related compounds such as P5P exert their antihypertensive effect is not fully understood, there are some possible explanations. The antihypertensive properties of vitamin B6 related compounds observed with diabetic individuals may be the result of improved insulin sensitivity and the concomitant normalization of blood glucose and lipid levels. Hyperglycemia and hyperlipidemia are both known to contribute to increased peripheral vascular resistance. Hypercholesterolemia may result in vascular endothelial injury (increased endothelial superoxide production, increased degradation of nitric oxide) and consequently impaired endothelium-dependent vasodilation. Hyperglycemia may contribute to vasoconstriction. High glucose concentrations may inhibit nitric oxide production and alter ion transport (i.e. increased sodium-hydrogen antiport activity) in vascular smooth muscle to favor vasoconstriction. The present inventors have now found that vitamin B6 related compounds are useful for treating diabetic hypertension by simultaneously increasing insulin sensitivity while nonnalizing blood glucose and lipid levels.

The enhanced antihypertensive activity observed with the coadministration of a vitamin B6 related compound and an ACE inhibitor may be due in part to the vitamin B6 related compound's role as co-factor in the various metabolic reactions in the renin-angiotensin system. In the diabetic state, energy is supplied mainly by amino acids and fat. Pyridoxal phosphate dependent enzymes, which are highly involved in amino acid metabolism, are important regulators of systemic blood pressure. Also, angiotensin II is metabolized by prolylcarboxypeptidase to angiotensin, a compound that does not cause vasoconstriction, or aldosterone release. Prolylcarboxypeptidase cleaves only peptides with penultimate proline residues, such as angiotensin II, and may therefore be involved in terminating signal transduction by peptide inactivation. Since prolylcarboxypeptidase also is responsible for generation of bradykinin, this system may serve as a physiologic counterbalance to the plasma renin-angiotensin system (RAS) by lowering blood pressure and preventing thrombosis. P5P may be a cofactor for prolylcarboxypeptidase activity. In light of these discoveries, embodiments of the invention include methods of treating a diabetic patient comprising the administration of a therapeutically effective amount of an ACE inhibitor and a vitamin B6 related compound. Administration of an ACE inhibitor and a vitamin B6 related compound positively influences insulin sensitivity, glucose control, endothelial function, and vascular function for the treatment of diabetes and diabetic hypertension. Methods of treatment of the present invention are more effective than currently available therapies for reducing blood pressure in diabetics with hypertension. Diabetic complications, which are aggravated by hypertension and vascular damage (e.g., retinopathy), are also expected to be treatable using methods of the present invention. The anti-nephropathic effects of vitamin B6 related compounds and of ACE inhibitors are also found to be enhanced when the two classes of agents were co-administered to diabetic individuals.

It is to be understood that this invention is not limited to specific dosage forms, carriers, or the like, and as such may vary. It is also to be understood that the terminology used herein is for the purpose of describing particular embodiments only, and is not intended to be limiting.

The 3-acylated analogue of pyridoxal includes: 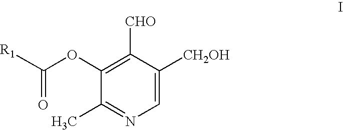
wherein,
R₁ is alkyl,
   alkenyl,
      in which alkyl or alkenyl
         can be interrupted by nitrogen, oxygen, or sulfur, and can be substituted at the terminal carbon by hydroxy, alkoxy, alkanoyloxy, alkanoyloxyaryl, alkoxyalkanoyl, alkoxycarbonyl, or dialkylcarbamoyloxy;
   alkoxy;
   dialkylamino;
   alkanoyloxy;
   alkanoyloxyaryl;
   alkoxyalkanoyl;
   alkoxycarbonyl;
   dialkylcarbamoyloxy; or
   aryl, in which aryl can be substituted by alkyl, alkoxy, amino, hydroxy, halo, nitro, or alkanoyloxy
   aryloxy,
   arylthio, or
   aralkyl, or a pharmaceutically acceptable acid addition salt thereof.
R₁ is a straight or branched alkyl group, a straight or branched alkenyl group, in which an alkyl or alkenyl group may be interrupted by a nitrogen or oxygen atom; an alkoxy group; a dialkylamino group; or an unsubstituted or substituted aryl group.

The term “alkyl” group includes a straight or branched saturated aliphatic hydrocarbon chain having from 1 to 8 carbon atoms, such as, for example, methyl, ethyl, propyl, isopropyl (1-methylethyl), butyl, tert-butyl (1,1-dimethylethyl), and the like.

The term “alkenyl” group includes an unsaturated aliphatic hydrocarbon chain having from 2 to 8 carbon atoms, such as, for example, ethenyl, 1-propenyl, 2-propenyl, 1-butenyl, 2-methyl-1-propenyl, and the like.

The above alkyl or alkenyl groups may optionally be interrupted in the chain by a heteroatom, such as, for example, a nitrogen or oxygen atom, forming an alkylaminoalkyl or alkoxyalkyl group, for example, methylaminoethyl or methoxymethyl, and the like.

The term “alkoxy” group includes an alkyl group as defined above joined to an oxygen atom having preferably from 1 to 4 carbon atoms in a straight or branched chain, such as, for example, methoxy, ethoxy, propoxy, isopropoxy (1-methylethoxy), butoxy, tert-butoxy (1,1-dimethylethoxy), and the like.

The term “dialkylamino” group includes two alkyl groups as defined above joined to a nitrogen atom, in which the alkyl group has preferably 1 to 4 carbon atoms, such as, for example, dimethylamino, diethylamino, methylethylamino, methylpropylamino, diethylamino, and the like.

The term “aryl” group includes an aromatic hydrocarbon group, including fused aromatic rings, such as, for example, phenyl and naphthyl. Such groups may be unsubstituted or substituted on the aromatic ring by, for example, an alkyl group of 1 to 4 carbon atoms, an alkoxy group of 1 to 4 carbon atoms, an amino group, a hydroxy group, or an acetyloxy group.

Preferred R₁ groups for compounds of formula I are toluyl or naphthyl. Such R₁ groups when joined with a carbonyl group form an acyl group 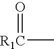
which preferred for compounds of formula I include toluoyl or β-naphthoyl. Of the toluoyl group, the p-isomer is more preferred.

Examples of 3-acylated analogues of pyridoxal include, but are not limited to, 2-methyl-3-toluoyloxy-4-formyl-5-hydroxymethylpyridine and 2-methyl-β-naphthoyloxy-4-formyl-5 -hydroxymethylpyridine

The 3-acylated analogue of pyridoxal-4,5-aminal includes: 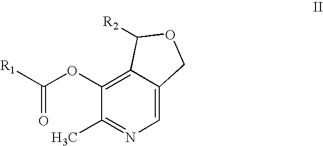
wherein,
R₁ is alkyl,
   alkenyl,
      in which alkyl or alkenyl
         can be interrupted by nitrogen, oxygen, or sulfur, and can be substituted at the terminal carbon by hydroxy, alkoxy, alkanoyloxy, alkanoyloxyaryl, alkoxyalkanoyl, alkoxycarbonyl, or dialkylcarbamoyloxy;
   alkoxy;
   dialkylamino;
   alkanoyloxy;
   alkanoyloxyaryl;
   alkoxyalkanoyl;
   alkoxycarbonyl;
   dialkylcarbamoyloxy; or
   aryl, in which aryl can be substituted by alkyl, alkoxy, amino, hydroxy, halo, nitro, or alkanoyloxy
   aryloxy,
   arylthio, or
   aralkyl; and

R₂ is a secondary amino group, or a pharmaceutically acceptable acid addition salt thereof.

R₁ is a straight or branched alkyl group, a straight or branched alkenyl group, in which an alkyl or alkenyl group may be interrupted by a nitrogen or oxygen atom; an alkoxy group; a dialkylamino group; or an unsubstituted or substituted aryl group; and

R₂ is a secondary amino group.

The terms “alkyl,” “alkenyl,” “alkoxy,” “dialkylamino,” and “aryl” are as defined above.

The term “secondary amino” group includes a group of the formula III: 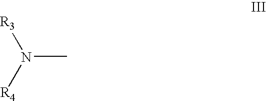
derived from a secondary amine R₃R₄NH, in which R₃ and R₄ are each independently alkyl, alkenyl, cycloalkyl, aryl, or, when R₃ and R₄ are taken together, may form a ring with the nitrogen atom and which may optionally be interrupted by a heteroatom, such as, for example, a nitrogen or oxygen atom. The terms “alkyl,” “alkenyl,” and “aryl” are used as defined above in forming secondary amino groups such as, for example, dimethylamino, methylethylamino, diethylamino, dialkylamino, phenylmethylamino, diphenylamino, and the like.

The term “cycloalkyl” refers to a saturated hydrocarbon having from 3 to 8 carbon atoms, preferably 3 to 6 carbon atoms, such as, for example, cyclopropyl, cyclopentyl, cyclohexyl, and the like.

When R₃ and R₄ are taken together with the nitrogen atom, they may form a cyclic secondary amino group, such as, for example, piperidino, and, when interrupted with a heteroatom, includes, for example, piperazino and morpholino.

Preferred R₁ groups for compounds of formula II include toluyl, e.g., p-toluyl, naphthyl, tert-butyl, dimethylamino, acetylphenyl, hydroxyphenyl, or alkoxy, e.g., methoxy. Such R₁ groups when joined with a carbonyl group form an acyl group 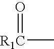
which preferred for compounds and formula II include toluoyl, β-naphthoyl, pivaloyl, dimethylcarbamoyl, acetylsalicyloyl, salicyloyl, or alkoxycarbonyl. A preferred secondary amino group may be morpholino.

Examples of 3-acylated analogues of pyridoxal-4,5-aminal include, but are not limited to, 1-morpholino-1,3-dihydro-7-(p-toluoyloxy)-6-methylfuro(3,4-c)pyridine; 1-morpholino-1,3-dihydro-7-(β-naphthoyloxy)-6-methylfuro(3,4-c)pyridine; 1-morpholino-1,3-dihydro-7-pivaloyloxy-6-methylfuro(3,4-c)pyridine; 1-morpholino-1,3-dihydro-7-carbamoyloxy-6-methylfuro(3,4-c)pyridine; and 1-morpholino-1,3-dihydro-7-acetylsalicyloxy-6-methylfuro(3,4-c)pyridine.

The compounds of formula I may be prepared by reacting pyridoxal hydrochloride with an acyl halide in an aprotic solvent. A suitable acyl group is 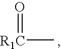
wherein R₁ is as defined above. A particularly suitable acyl halide includes p-toluoyl chloride or β-naphthoyl chloride. A suitable aprotic solvent includes acetone, methylethylketone, and the like.

The compounds of formula II may be prepared by reacting 1-secondary amino-1,3-dihydro-7-hydroxy-6-methylfuro(3,4-c)pyridine with an acyl halide in an aprotic solvent. An acyl group is 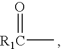
wherein R₁ is as defined above. A particularly suitable acyl halide includes p-toluoyl chloride, β-naphthoyl chloride, trimethylacetyl chloride, dimethylcarbamoyl chloride, and acetylsalicyloyl chloride. A particularly suitable secondary amino group includes morpholino.

The compound 1-morpholino-1,3-dihydro-7-hydroxy-6-methylfuro(3,4-c)pyridine may be prepared by methods known in the art, for example, by reacting morpholine and pyridoxal hydrochloride at a temperature of about 100° C. in a solvent. A suitable solvent includes, for example, toluene. Similarly, other secondary amines as defined for R₂ may be used as reactants to prepare the appropriate 1-secondary amino compounds.

The compounds of formula I may alternatively be prepared from the compounds of formula II by reacting a compound of formula II with an aqueous acid, such as, for example, aqueous acetic acid.

The pyridoxine phosphate analogue includes: 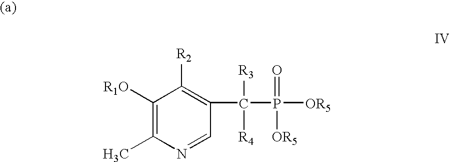
wherein,
R₁ is hydrogen or alkyl;
R₂ is —CHO—, —CH₂OH, —CH₃, —CO₂R6 in which R6 is hydrogen, alkyl, aryl; or
R₂ is —CH₂—O alkyl in which alkyl is covalently bonded to the oxygen at the 3-position instead of R₁;
R₃ is hydrogen and R₄ is hydroxy, halo, alkoxy, alkanoyloxy, alkylamino, or arylamino; or
R₃ and R₄ are halo; and
R₅ is hydrogen, alkyl, aryl, aralkyl, or —CO₂R₇ in which R₇ is hydrogen, alkyl, aryl, or aralkyl; 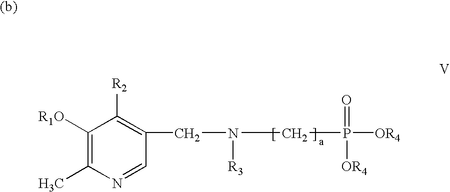
   wherein,
R₁ is hydrogen or alkyl;
R₂ is —CHO, —CH₂OH, —CH₃, —CO₂R₅ in which R₅ is hydrogen, alkyl, aryl; or
R₂ is —CH₂—O alkyl in which alkyl is covalently bonded to the oxygen at the 3-position instead of R₁;
R₃ is hydrogen, alkyl, aryl, aralkyl,
R₄ is hydrogen, alkyl, aryl, aralkyl, or —CO₂R6 in which R6 is hydrogen, alkyl, aryl or aralkyl;
n is 1 to 6; and 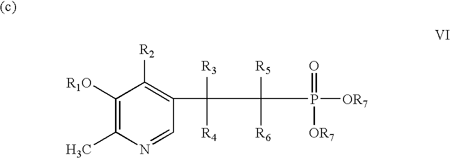
   wherein,
R₁ is hydrogen or alkyl;
R₂ is —CHO—, CH₂OH—, —CH₃, —CO₂R₈ in which R₈ is hydrogen, alkyl, aryl; or
R₂ is —CH₂—O alkyl- in which alkyl is covalently bonded to the oxygen at the 3-position instead of R₁;
R₃ is hydrogen and R₄ is hydroxy, halo, alkoxy, or alkanoyloxy; or
R₃ and R4 can be taken together to form ═O;
R₅ and R6 are hydrogen; or
R₅ and R6 are halo;
R₇ is hydrogen, alkyl, aryl, aralkyl, or —CO₂R₈ in which R₈ is hydrogen, alkyl, aryl, or aralkyl.

Some of the compounds described herein contain one or more asymmetric centers and this may give raise to enantiomers, diasteriomers, and other stereroisomeric forms which may be defined in terms of absolute stereochemistry as (R)- or (S)-. The present invention is meant to include all such possible diasteriomers and enantiomers as well as their racemic and optically pure forms. Optically active (R)- and (S)-isomers may be prepared using chiral synthons or chiral reagents, or resolved using conventional techniques. When the compounds described herein contain olefinic double bonds or other centers of geometric symmetry, and unless specified otherwise, it is intended that the compounds include both E and A geometric isomers. Likewise all tautomeric forms are intended to be included.

Pharmaceutically acceptable acid addition salts of the compounds suitable for use in methods of the invention include salts derived from nontoxic inorganic acids such as hydrochloric, nitric, phosphoric, sulfuric, hydrobromic, hydriodic, hydrofluoric, phosphorous, and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate, n-methyl glutamine, etc. (see, e.g., Berge et al., J. Pharmaceutical Science, 66: 1-19 (1977).

The acid addition salts of the basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

A medical professional readily determines a subject who is exhibiting symptoms of any one or more of the diseases described herein. Regardless of the route of administration selected, compounds suitable for use in the methods described herein are formulated into pharmaceutically acceptable unit dosage forms by conventional methods known to the pharmaceutical art. An effective but nontoxic quantity of the compound is employed in treatment. The compounds can be administered in enteral unit dosage forms, such as, for example, tablets, sustained release tablets, enteric coated tablets, capsules, sustained release capsules, enteric coated capsules, pills, powders, granules, solutions, and the like. They may also be administered parenterally, such as, for example, subcutaneously, intramuscularly, intradermally, intramammarally, intravenously, and other administrative methods known in the art.

Although it is possible for a compound suitable for use in methods described herein to be administered alone in a unit dosage form, preferably the compound is administered in admixture as a pharmaceutical composition suitable for use in methods of the invention. A pharmaceutical composition comprises a pharmaceutically acceptable carrier and a compound. A pharmaceutically acceptable carrier includes, but is not limited to, physiological saline, ringers, phosphate buffered saline, and other carriers known in the art. Pharmaceutical compositions may also include additives, for example, stabilizers, antioxidants, colorants, excipients, binders, thickeners, dispersing agents, readsorpotion enhancers, buffers, surfactants, preservatives, emulsifiers, isotonizing agents, and diluents. Pharmaceutically acceptable carriers and additives are chosen such that side effects from the pharmaceutical compound are minimized and the performance of the compound is not canceled or inhibited to such an extent that treatment is ineffective.

Methods of preparing pharmaceutical compositions containing a pharmaceutically acceptable carrier and a compound suitable for use in methods of the invention are known to those of skill in the art. All methods may include the step of bringing the compound in association with the carrier and additives. In general, the formulations are prepared by uniformly and intimately bringing the compound of the invention into association with a liquid carrier or a finely divided solid carrier or both, and then, if necessary, shaping the product into the desired unit dosage form.

Examples of ACE inhibitors useful for practicing the methods of treatment according to the present invention include but are not limited to: benazepril; captopril; cilazapril; enalapril; enalaprilat; fosinopril; lisinopril; moexipril; perindopril; quinapril; ramipril; trandolapril; or a mixture thereof. In a preferred embodiment, the ACE inhibitor is lisinopril. In a further preferred embodiment of the invention, the ACE inhibitor component administered is lisinopril and the vitamin B6 related component administered is P5P. In one aspect, the invention provides a method of improving metabolic function in a diabetic patient in need thereof comprising the administration of an ACE inhibitor and a vitamin B6 related compound. The metabolic function to be improved in the diabetic patient may include, but is not limited to: increased insulin sensitivity, increased glycemic control including decreased levels of HbA1c, decreased insulinemia, decreased hyperglycemia, and decreased hyperlipidemia including decreased levels of low density lipoprotein (LDL) and/or increased levels of high density lipoprotein (HDL). The metabolic effects of vitamin B6 related compounds and of ACE inhibitors are also found to be enhanced when the two classes of agents were co-administered to diabetic individuals.

In a further aspect, the invention provides a method of improving vascular function in a diabetic patient in need thereof comprising administering a therapeutically effective amount of an ACE inhibitor and a vitamin B6 related compound. Improvement of vascular function includes prevention or the amelioration of damage to either the macrovasculature system or the microvasculature system. Improvement of vascular function includes prevention or treatment of cardiovascular disease associated with diabetes. Examples of cardiovascular diseases which may be prevented or treated with pharmaceutical compositions according to the invention include but are not limited to: peripheral vascular disease, atherothrombosis, and atherosclerosis. The improvement of vascular function also includes the prevention or treatment of renal failure and in particular damage to the renal vasculature system resulting from diabetic complications. In a preferred embodiment, methods are useful for prevention and treatment of nephropathy. Improvement of vascular function further includes prevention and treatment of damage to the vasculature system in the eye resulting from diabetic complications. In a preferred embodiment, methods are useful for the prevention and treatment of retinopathy. Vascular effects of vitamin B6 related compounds and of ACE inhibitors are also found to be enhanced when the two classes of agents were co-administered to diabetic individuals.

In a yet a further aspect, the present invention provides a method of improving endothelial function in a diabetic patient in need thereof comprising administering a therapeutically effective amount of an ACE inhibitor and a vitamin B6 related compound. Improvement of endothelial function includes prevention and treatment of damage to endothelium caused by diabetic related metabolic disorders. Examples of endothelial dysfunction include but are not limited to atherogenesis. Endothelial effects of vitamin B6 related compounds and of ACE inhibitors are also found to be enhanced when the two classes of agents were co-administered to diabetic individuals.

In a still further aspect, the present invention provides a method of treating or inhibiting hypertension in a diabetic patient in need thereof comprising administering an ACE inhibitor and a vitamin B6 related compound. It will be appreciated that the hypertension may be primary hypertension or a secondary hypertension. In a preferred embodiment of the invention, the hypertension to be treated is “diabetic hypertension” resulting from metabolic (such as poor insulin sensitivity and poor glycemic control), vascular and/or endothelial dysfunction in the diabetic patient. In a further preferred embodiment of the invention, a diabetic patient treated is an individual with type 2 diabetes.

Embodiments of the present invention reduce mean daytime ambulatory systolic blood pressure (MDASBP) and mean daytime ambulatory diastolic blood pressure (MDADBP). A combinations of pyridoxal-5′-phosphate and an ACE inhibitor provide a greater reduction of MDASBP and MDADBP from baseline compared to said ACE inhibitor alone. Surprisingly, a combination of 300 mg pyridoxal-5′-phosphate (P5P) and 20 mg lisinopril per dose further reduced MDASBP and MDADBP from baseline compared to 1000 mg pyridoxal-5′-phosphate and 20 mg lisinopril per dose. A 300/20 (P5P/lisinopril) mg/dose also further reduced fasting serum glucose, glycated hemoglobin (HbA1c), and triglycerides from baseline compared to a 1000/20 (P5P/lisinopril) mg/dose.

Preferably, ACE inhibitors and vitamin B6 related compounds are administered orally. Preferred oral dosage forms contain a therapeutically effective unit dose of each active agent, wherein the unit dose is suitable for once-daily oral administration. The therapeutic effective unit dose of any of the active agents will depend on a number of factors. In particular these factors include, but are not limited to, the identity of the compounds to be administered, the formulation, the route of administration employed, the patient's gender, age, and weight, the severity of the condition being treated, and the presence of concurrent illness affecting the gastro-intestinal tract, the hepatobiliary system, and the renal system. Methods for determining dosage and toxicity are well known, with studies generally beginning in animals and then progressing to humans if no significant animal toxicity is observed. Appropriateness of a dosage can be assessed by monitoring the following, but not limited to: antihypertensive efficacy (mean decrease in daytime systolic ambulatory blood pressure), metabolic function (for example, insulinemia, fasting serum glucose, glycated hemoglobin, and triglycerides), endothelial function (for example, ICAM-1, VCAM-1, E-selectin and albuminuria), inflammatory marker CRP, homocysteine, and creatinine. Where a dose does not improve metabolic, vascular and/or endothelial function or reduce blood pressure following at least 2 to 4 weeks of treatment, the dose can be increased.

A therapeutic effective unit dose of an ACE inhibitor will vary depending on the particular ACE inhibitor employed. Suitable dosage ranges for ACE inhibitors are known. Where an ACE inhibitor is lisinopril, a preferred unit dosage is between 5 and 40 mg/day and more preferably, 20 mg/day. Where an ACE inhibitor is captopril, a preferred unit dosage is between 25 and 150 mg/day. Where the ACE inhibitor is enalapril, a preferred unit dosage is between 5 and 40 mg/day. Where the ACE inhibitor is ramipril, a preferred unit dosage is between 1.25 and 10 mg/day. Where the ACE inhibitor is trandolapril, a preferred unit dosage is between 1 and 4 mg/day.

A therapeutic effective unit dose of a vitamin B6 related compound is preferably between 1 and 1000 mg/day. Where the vitamin B6 related compound employed is P5P, a therapeutic effective unit dose is preferably between 100 and 1000 mg/day. Dosage ranges of pyridoxal-5′-phosphate typically are about 1 to about 1000 mg/day, about 100 to about 750 mg/day, about 100 to about 500 mg/day, about 100 to about 300 mg/day, about 200 to about 300 mg/day, about 200 to about 275 mg/day, about 225 to about 325 mg/day, about 225 to about 300 mg/day, about 225 to about 400 mg/day, about 225 to about 500 mg/day, about 225 to about 275 mg/day, about 230 to about 270 mg/day, about 240 to about 260 mg/day, and about 245 to about 255 mg/day. A dosage of about 250 mg/day and about 300 mg/day of pyridoxal-5′-phosphate is also typical.

Although the present invention has been described with reference to illustrative embodiments, it is to be understood that the invention is not limited to these precise embodiments, and that various changes and modifications may be effected therein. All such changes and modifications are intended to be encompassed in the appended claims.

### EXAMPLES

### Example 1

### Animal Toxicology Studies of Pyridoxal-5′-Phosphate (P5P)

As a prelude to human clinical studies, the toxicology of P5P was assessed by conventional means using two animal species, rats and dogs. Acute toxicity evaluations indicated no significant toxicity at doses up to 5 g/kg in the rat and 100 mg/kg in dogs. Rats administered P5P orally at 50 mg/kg for 14 days showed no signs of toxicity. Long term studies, 13-week oral toxicity in dogs, and 26-week oral toxicity in rats, were completed. In the 13-week dog study, no drug related toxicities were observed at both 10 and 25 mg/kg. With the exception of anorexia and body weight loss in the high dose 50-60 mg/kg dose group, all other findings were considered to be mild to moderate. During the recovery phase, the 50-60 mg/kg group animals recovered almost completely. No findings of toxicological significance were observed at any dose level (50, 100/175, 175/325 mg/kg) in the 26-week rat toxicity study, other than reversible reduction in body weight gain and increased incidence of stomach microulcers in the high dose group.

### Example 2

### Phase I Tolerance Study of Pyridoxal-5′-Phosphate (P5P)

In a Phase I single dose tolerance study, conducted in accordance with generally accepted clinical practice standards, groups of six patients were tested at 15 mg/kg, 30 mg/kg, and 60 mg/kg (enteric coated tablets). No adverse events were reported in the 15 mg/kg dose group. One subject in the 30 mg/kg dose group experienced events of dizziness and sleepiness. Four subjects in the 60 mg/kg dose group reported a total of 10 adverse events including diarrhea, bradycardia, bubbly stomach, flatulence, and headaches that were mild in severity. During the Phase I multi-dose tolerance study, five of six patients treated with 30 mg/kg P5P tolerated the medication well, while one patient withdrew from the trial due to vomiting and diarrhea. An evaluation of multidose tolerance at 60 mg/kg resulted in all 6 treated patients experiencing a variety of mild gastrointestinal symptoms considered to be probably related to study drug. Pharmacokinetics and statistical analyses did not demonstrate dose-linearity but the small numbers of subjects enrolled at each dose-level and the large inter-subject variability could have contributed to this observation.

### Example 3

### Phase II Clinical Study: Effectiveness of Pyridoxal-5′-Phosphate (P5P) in Diabetic Patients

In a phase II clinical study, conducted in accordance with generally accepted clinical practice standards, diabetic hypertensive patients were treated with P5P. Glucose control was determined by measuring glycated hemoglobin levels (HbA1c). Four weeks prior to treatment, patients ceased all antihypertensive therapy. Following the washout period, baseline HbA1c measurements were taken. Patients were than treated with 250 mg, 500 mg, and 750 mg of P5P for two weeks at each dosage. P5P treatment was then discontinued for 4 weeks. Following the washout period, HbA1c measurements were taken. Patients who presented with clinically elevated HbA1c at the start of the treatment and who completed the treatment with P5P were found to show a 5.4% reduction in HbA1c levels as compared to baseline.

### Example 4

### Phase II Clinical Study: Effectiveness of Pyridoxal-5′-Phosphate (P5P) and Lisinopril in Diabetic Patients

Objective—A phase II clinical study was conducted to determine the effects of pyridoxal-5′-phosphate in combination with lisinopril on blood pressure and metabolic function in hypertensive patients with type 2 diabetes.

Summary of Study Design—The phase II study was a randomized, parallel group, cross-over, double-blinded to study medication, placebo-controlled comparison of P5P BID at total daily doses of 100, 300 or 1000 mg alone and in combination with 20 mg lisinopril given once daily (QD). In order to protect against antihypertensive and metabolic carry-over effects of lisinopril, 160 patients were randomized in 2 different treatment sequences. Patients randomized in the first treatment sequence received an 8-week treatment with lisinopril 20 mg and P5P (or placebo) and then an 8-week treatment with P5P alone (or placebo). Patients randomized in the second treatment sequence received an 8-week treatment with P5P alone and then an 8-week treatment with lisinopril 20 mg and P5P (or placebo). In each treatment sequence, all patients were randomized to P5P at the different prespecified dosages.

Mean trough sitting and standing BP were measured at each visit. Twenty-four hour ambulatory BP monitoring (ABPM) was performed at Visit 2 prior to randomization (end of washout period) and after week 8 (Visit 5) and week 16 (Visit 8) weeks of active therapy. Laboratory tests were performed at screening (Visit 1), prior to randomization (Visit 2), at week 2 (Visit 3a), week 8 (Visit 5), week 10 (Visit 6a), and at week 16 (Visit 8).

A physical examination and an electrocardiogram were performed at screening (Visit 1) and at the end of the study (Visit 8).

Patients with a mean trough sitting systolic blood pressure (SiSBP) greater than (>) 180 mmHg at anytime following randomization had measurements repeated within 24 hours. If the mean trough SiSBP was greater than 180 mmHg at the following visit, the patient was discontinued from the study and appropriate therapy was instituted.

Patients with a mean trough sitting diastolic blood pressure (SiDBP) greater than 110 mmHg at anytime during the study had measurements repeated within 24 hours. If the mean trough SiDBP remained greater than 110 mmHg, then the patient was discontinued from the study and appropriate therapy was instituted.

Patients with a mean trough SiSBP of greater than 160 mmHg four (4) weeks after randomization had measurements repeated within 48 hours. If the mean trough SiSBP was greater than 160 mmHg at the following visit, the patient was discontinued from the study and appropriate therapy was instituted. These patients were part of the safety evaluation.

Patients with a mean trough SiDBP of 105 mm Hg four (4) weeks after randomization had measurements repeated within 48 hours. If the mean trough SiDBP was greater than 105 mmHg at the following visit, the patient was discontinued from the study and appropriate therapy was instituted. These patients were part of the safety evaluation.

Treatment Plan—Two to Four-week Washout (Baseline) Period: Patients were instructed on the proper procedure for discontinuing their current antihypertensive medications (discontinuation or tapering) according to the manufacturer's label specifications. If a patient's antihypertensive treatment needed to be tapered earlier, the Investigator complied with the corresponding timelines before randomization. With the exception of any tapering off of prior therapy, if any, no other anti-hypertensive medication was given to the patient during the washout period. Patients continued any existing diabetic treatment with sulfonylureas (tolbutamide, tolazamide, acetohexamide, chlorpropamide and second generation glyburide, glipizide, glimepiride), D-phenylalanine derivatives, metformin, thiazolidinediones, acarbose, miglitol, and/or insulin throughout the study. Patients received placebo to be taken twice daily during the washout period. Standard diabetic medication was maintained throughout study. The duration of the washout period was two to four weeks, at the discretion of the Investigator taking into consideration whether the patient's blood pressure had stabilized following removal of any prior antihypertensive medication.

Active (Study) Medication Period. After the washout period, eligible patients were randomized to one of the 2 following sequences of treatment for 16 weeks.
Week 0 to 8: Treatment period (P5P alone (or placebo) or P5P (or placebo) and Lisinopril)
Week 8 to 16: Treatment period (P5P alone (or placebo) or P5P (or placebo) and Lisinopril)

Study Groups—The patients were randomized into one of four groups:

Group A)

| | |
|---|---|
| Sequence 1 | Placebo and then Placebo + Lisinopril 20 mg |
| Sequence 2 | Placebo + Lisinopril 20 mg and then Placebo alone |

Group B)

| | |
|---|---|
| Sequence 1 | P5P 100 mg and then P5P 100 mg + Lisinopril 20 mg |
| Sequence 2 | P5P 100 mg + Lisinopril 20 mg and then P5P 100 mg alone |

Group C)

| | |
|---|---|
| Sequence 1 | P5P 300 mg and then P5P 300 mg + Lisinopril 20 mg |
| Sequence 2 | P5P 300 mg + Lisinopril 20 mg and then P5P 300 mg alone |

Group D)

| | |
|---|---|
| Sequence 1 | P5P 1000 mg and then P5P 1000 mg + Lisinopril 20 mg |
| Sequence 2 | P5P 1000 mg + Lisinopril 20 mg and P5P 1000 mg alone |

All medications were taken at the same time each day during washout and treatment periods:

| | |
|---|---|
| P5P/placebo: | morning dose: 7:00 am to 11:00 am |
| | evening dose: 7:00 pm to 11:00 pm |
| Lisinopril: | 7:00 am to 11:00 am (with P5P/placebo morning dose) |

On the day of a clinic visit, all study medication for that morning were taken following the completion of all the scheduled study parameters.

Efficacy Measurements—Blood pressure was measured using a sphygmomanometer maintained in good condition (standard mercury, Bp-Thru, Omron) to measure blood pressure. Care was taken to use the proper cuff size. Blood pressure was measured in the sitting and standing positions at every clinic visit (baseline and treatment). If a mercury sphygmomanometer was used, Korotkoff Phase V (disappearance of sounds) was used as the criterion for diastolic blood pressure. The proper cuff size was used on the same arm throughout the study. The arm used for blood pressure measurement was recorded in the workbooks. The routine blood pressure measurement was a “trough” measurement; that is, the measurements were taken 24 hours (range 22 to 26 hrs) after the last morning dose. Trough measurements were taken at each clinic visit.

Ambulatory blood pressure (ABP) was measured using a SpaceLabs Medical ABPM Monitor Model 90207 (SpaceLabs Medical Inc., Redmond, Wash.). The ambulatory blood pressure measuring (ABPM) device was fitted to the subject on the morning of visit 2. Following the initiation of two manual readings, a third manual reading was initiated and began the 24-hour monitoring period. Subjects returned to the clinic the following day (Visit 3) arriving at least 15 minutes prior to the completion of the 24-hour monitoring period. A manual reading was initiated at the end of the 24-hour period to ensure that there was at least one data point in the last hour of the 24-hour period. Subjects were instructed to initiate a manual reading should they be late for their scheduled clinic appointment to ensure that a reading in the last hour of the 24-hour period was not missed. On completion of the readings, the ABPM device was removed from the subject. Data from the ABPM device were then be downloaded in the computer database. At baseline, the ABPM session had to be deemed successful and mean daytime ambulatory systolic BP had to be ≧135 mm Hg.

If at visit 3, the ABPM session was deemed unsuccessful, a repeat session was permitted within 72 hours.

In addition to baseline, ambulatory monitoring was repeated after 8 and 16 weeks of therapy to assess active treatment efficacy. If the ABPM session was deemed unsuccessful on either of these timepoints, a repeat session was permitted within 72 hours provided the patient maintained the same dosing regimen as immediately prior to the ABPM measurement in question.

Other Efficacy Endpoints—Co-primary endpoints of the study included the effect of the combination of pyridoxal-5′-phosphate and the ACE inhibitor, lisinopril, on blood pressure and metabolic functions. Changes in both mean daytime ambulatory systolic blood pressure (MDASBP) and mean daytime ambulatory diastolic blood pressure (MDADBP) were measured. Metabolic endpoints of the study included the effect on carbohydrate (fasting glucose (GLU), HbA1c) and lipid (triglycerides (TG), total cholesterol (TC), and LDL) metabolism, versus placebo. Samples were sent to a central laboratory for analysis.

Results—Subjects treated with pyridoxal-5′-phosphate and lisinopril had lowered blood pressure and improved metabolic function as evidenced by improved glucose control and improved lipid levels.

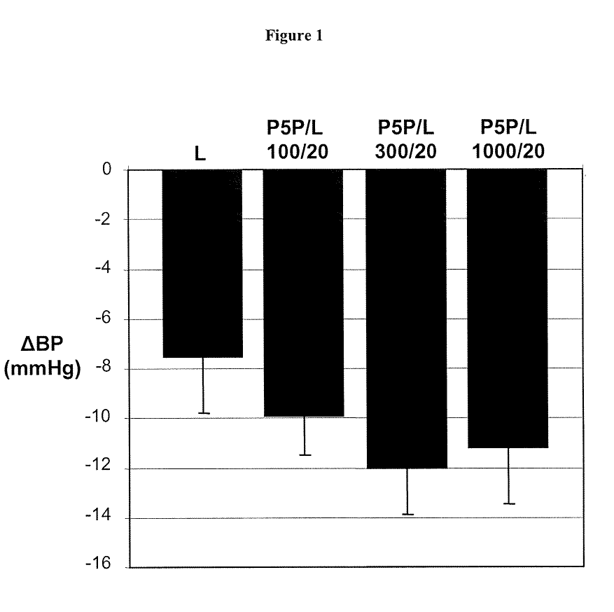

1. Blood Pressure. After correcting for placebo effects, the combination of pyridoxal-5′-phosphate and lisinopril induced a statistically significant reduction in MDASBP from baseline after eight weeks of therapy compared to a therapy of lisinopril and placebo (FIG. 1). In patients receiving lisinopril (20 mg/dose) and placebo, blood pressure was reduced by 7.5 mmHg after 8 weeks of treatment (p=0.002, thereby statistically significant since p<0.05). Eight weeks of administering a 20 mg/dose of lisinopril in combination with 100 mg/dose, 300 mg/dose, or 1000 mg/dose of pyridoxal-5′-phosphate further lowered patients' blood pressure from baseline by 9.9 mmHg, 12.0 mmHg, and 11.2 mmHg, respectively (p<0.001 for all three combinations).

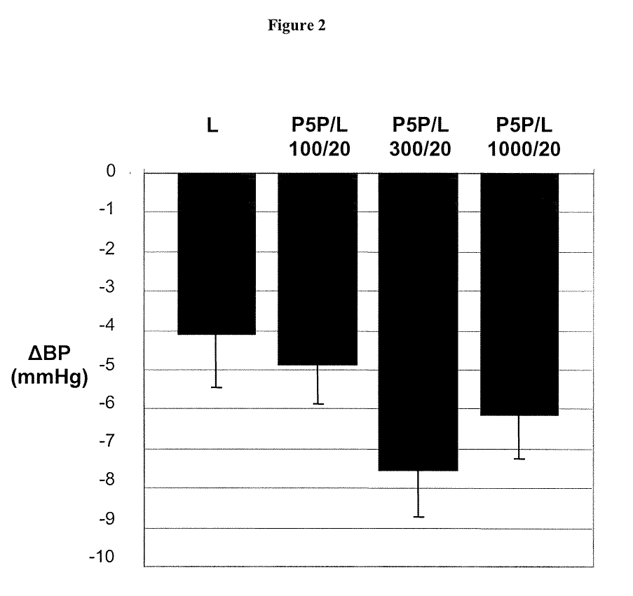

The same effect was observed for changes in MDADBP (FIG. 2). In patients receiving lisinopril (20 mg/dose) plus placebo, blood pressure was reduced by 4.1 mmHg after 8 weeks of treatment (p=0.005). Eight weeks of administering a 20 mg/dose of lisinopril in combination with 100 mg/dose, 300 mg/dose, or 1000 mg/dose of pyridoxal-5′-phosphate further lowered patients' blood pressure from baseline by 4.9 mmHg, 7.5 mmHg, and 6.2 mmHg, respectively (p<0.001 for all three combinations).

In this patient population, administration of the combination of lisinopril and pyridoxal-5′-phosphate significantly lowered both the MDASBP and MDADBP. Surprisingly, the 300/20 (P5P/L) mg/dose lowered blood pressures to a greater degree than either the lower dose (100/20 (P5P/L) mg/dose) or the higher dose (1000/20 (P5P/L) mg/dose) of the combination. The combination of lisinopril and pyridoxal-5′-phosphate lowered blood pressures to a greater degree than lisinopril in combination with the placebo.

2. Metabolic Functions. After the sixteen week study, patients administered lisinopril in combination with pyridoxal-5′-phosphate had improved carbohydrate and lipid metabolism compared to patients receiving lisinopril plus placebo.

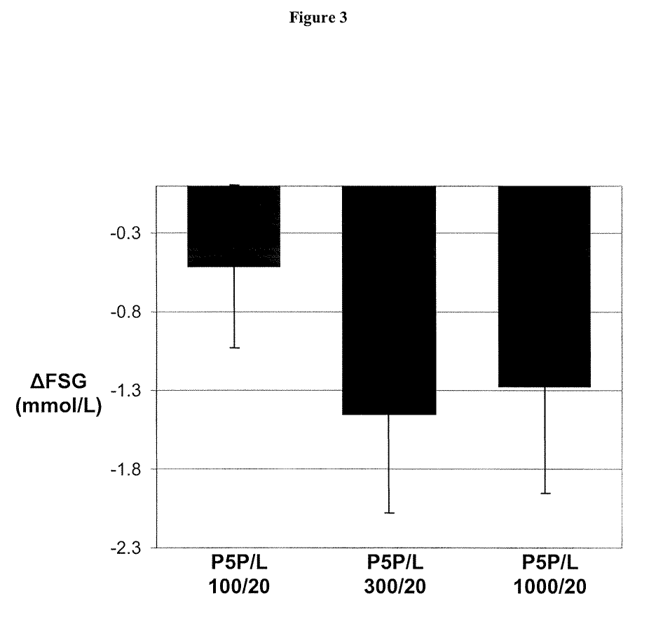

a. Fasting Serum Glucose. After correcting for placebo effects, the combination of pyridoxal-5′-phosphate and lisinopril further reduced fasting serum glucose from baseline after sixteen weeks of therapy (FIG. 3). After sixteen weeks of treatment, the 100 mg/dose in combination with 20 mg/dose of lisinopril (8 weeks with lisinopril and 8 weeks without), 300 mg/dose (8 weeks with lisinopril and 8 weeks without), or 1000 mg/dose(8 weeks with lisinopril and 8 weeks without) of pyridoxal-5′-phosphate further lowered patients' fasting serum glucose from baseline by 0.51 mmol/L (9.2 mg/dL, p=0.330), 1.45 mmol/L (26.1 mg/dL, p=0.030 thereby statistically significant since p<0.05), and 1.27 mmol/L (22.9 mg/dL, p=0.070), respectively, after subtracting out values for lisinopril administered alone (placebo).

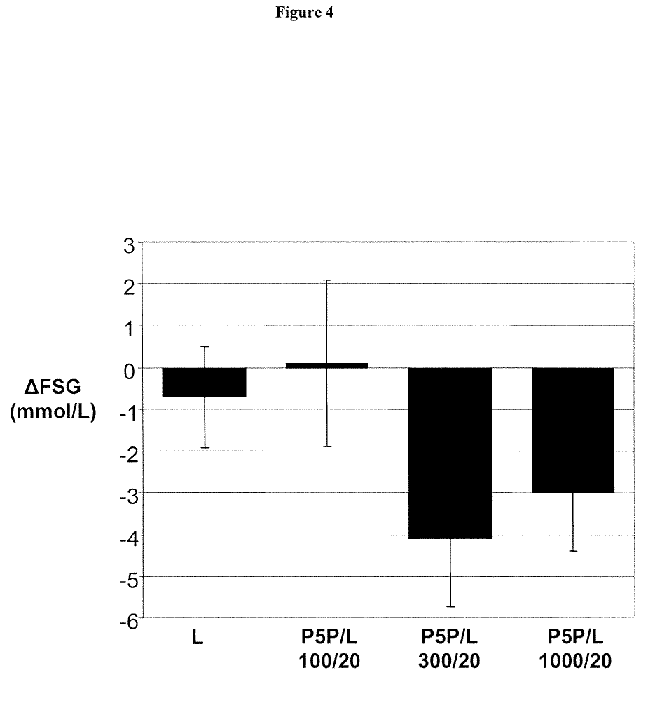

In patients with a baseline fasting serum glucose of 10 mmol/L or greater, the 300/20 mg/L and 100/20 mg/L doses were effective at lowering fasting serum glucose from baseline, but not the 100/20 mg/L dose of the combination of pyridoxal-5′-phosphate and lisinopril (FIG. 4). In patients receiving lisinopril (20 mg/dose) and placebo, fasting serum glucose was reduced by 0.7 mmol/L (12.6 mg/dL, p=0.589) after 16 weeks of treatment. After sixteen weeks of sequence 1 and sequence 2, administering a 20 mg/dose of lisinopril in combination with 300 mg/dose or 1000 mg/dose of pyridoxal-5′-phosphate further lowered patients' fasting serum glucose from baseline by 4.1 mmol/L (73.9 mg/dL, p=0.053) and 3.0 mmol/L (54.0 mg/dL, p=0.074), respectively. Patients receiving the 100/20 (P5P/L) mg/dose had a slight increase, 0.1 mmol/L (1.8 mg/dL), increase in serum fasting glucose.

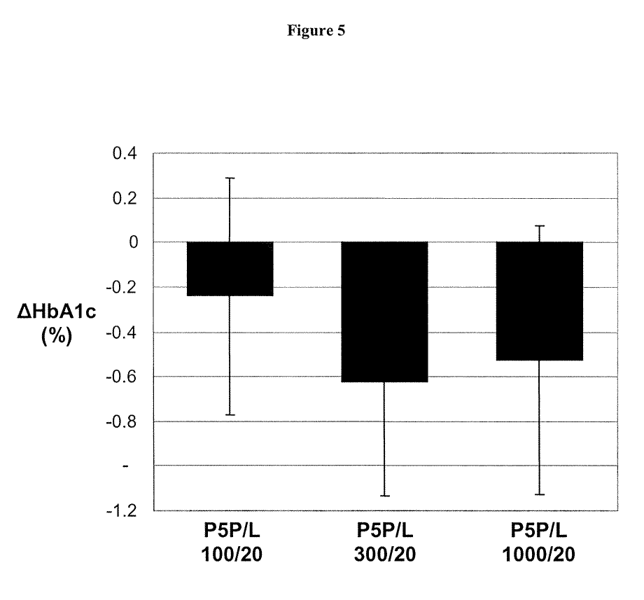

b. Glyeated Hemoglobin. After correcting for placebo effects, the combination of pyridoxal-5′-phosphate and lisinopril reduced glycated hemoglobin (HbA1c) from baseline after sixteen weeks of therapy in patients with a HbA1c of 8% or greater at baseline (FIG. 5). After sixteen weeks of sequence 1 and sequence 2, administering a 20 mg/dose of lisinopril in combination with 100 mg/dose, 300 mg/dose, or 1000 mg/dose of pyridoxal-5′-phosphate further lowered patients' HbA1c from baseline by 0.24% (n=7, p=0.657), 0.62% (n=4, p=0.269), and 0.52 (n=8, p=0.404), respectively, after subtracting out values for lisinopril administer alone (placebo).

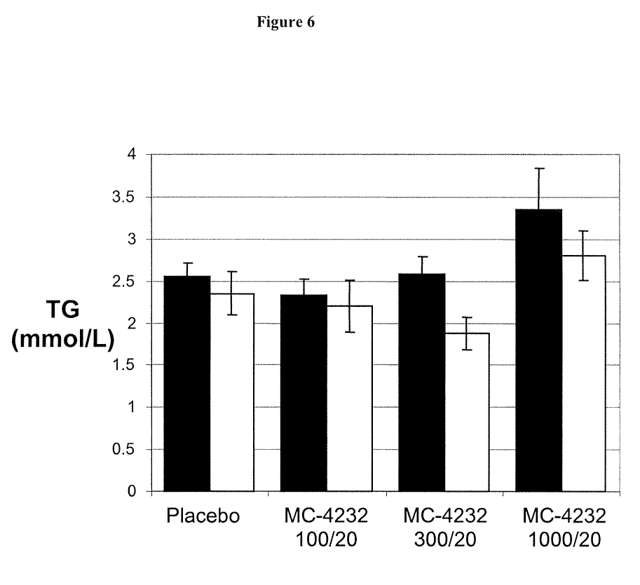

c. Triglycerides. The combination of pyridoxal-5′-phosphate and lisinopril reduced triglyceride levels (TG) from baseline after sixteen weeks of therapy compared to a therapy of lisinopril and placebo in patients with a triglyceride level of 1.7 mmol/L or greater at baseline (FIG. 6). In patients receiving lisinopril (20 mg/dose) and placebo, triglyceride levels were reduced by 0.22 mmol/L (19.6 mg/dL) after 16 weeks of treatment (p=0.367). After sixteen weeks of sequence 1 and sequence 2, administering a 20 mg/dose of lisinopril in combination with 100 mg/dose, 300 mg/dose, or 1000 mg/dose of pyridoxal-5′-phosphate further lowered patients' triglyceride levels from baseline by 0.13 mmol/L (11.6 mg/dL, p=0.599), 0.70 mmol/L (62.3 mg/dL, p=0.040 thereby statistically significant since p<0.05), and 0.56 mmol/L (49.8 mg/dL, p=0.116), respectively.

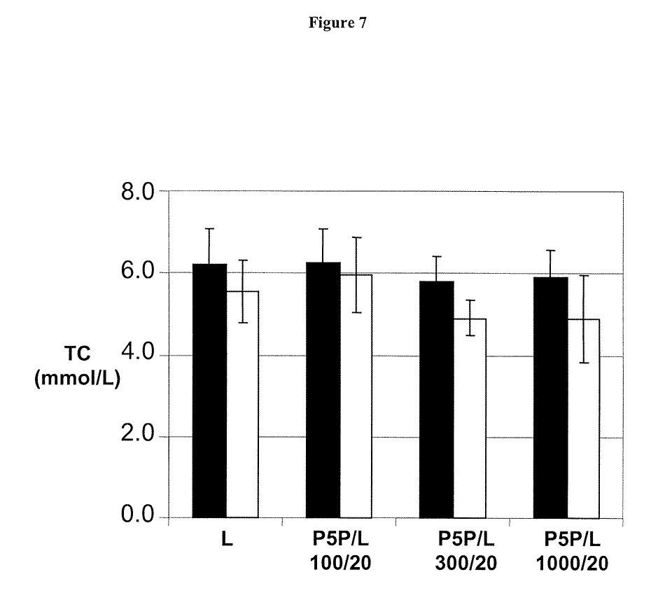

d. Cholesterol. The combination of pyridoxal-5′-phosphate and lisinopril reduced total cholesterol (TC) levels from baseline after sixteen weeks of therapy in patients with a total cholesterol level of 5.2 mmol/L at baseline (FIG. 7). In patients receiving lisinopril (20 mg/dose) and placebo, total cholesterol was reduced by 0.66 mmol/L (25.7 mg/dL) after 16 weeks of treatment (p=0.152). After sixteen weeks of sequence 1 and sequence 2, administering a 20 mg/dose of lisinopril in combination with 100 mg/dose, 300 mg/dose, or 1000 mg/dose of pyridoxal-5′-phosphate further lowered patients' triglyceride levels from baseline by 0.30 mmol/L (11.7 mg/dL, p=0.535), 0.90 mmol/L (35.1 mg/dL, p=0.019 thereby statistically significant since p<0.05), and 1.03 mmol/L (40.2 mg/dL, p=0.001 thereby statistically significant), respectively.

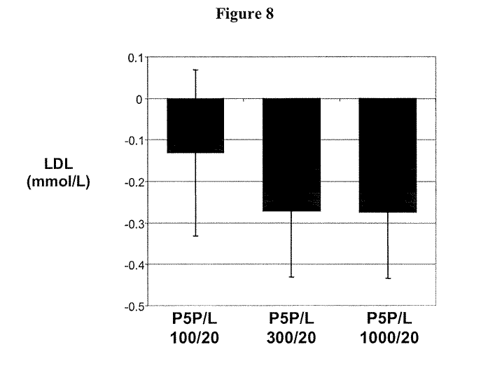

For low density lipoprotein cholesterol in particular, the combination of pyridoxal-5′-phosphate and lisinopril further reduced low density lipoproteins (LDL) from baseline after sixteen weeks of therapy (FIG. 8). After sixteen weeks of sequence 1 and sequence 2, administering a 20 mg/dose of lisinopril in combination with 100 mg/dose, 300 mg/dose, or 1000 mg/dose of pyridoxal-5′-phosphate further lowered patients' LDL levels from baseline by 0.13 mmol/L (5.1 mg/dL, p=0.520), 0.27 mmol/L (20.5 mg/dL, p=0.095), and 0.27 mmol/L (20.5 mg/dL, p=0.095), respectively, after subtracting out values for lisinopril administered alone (placebo).

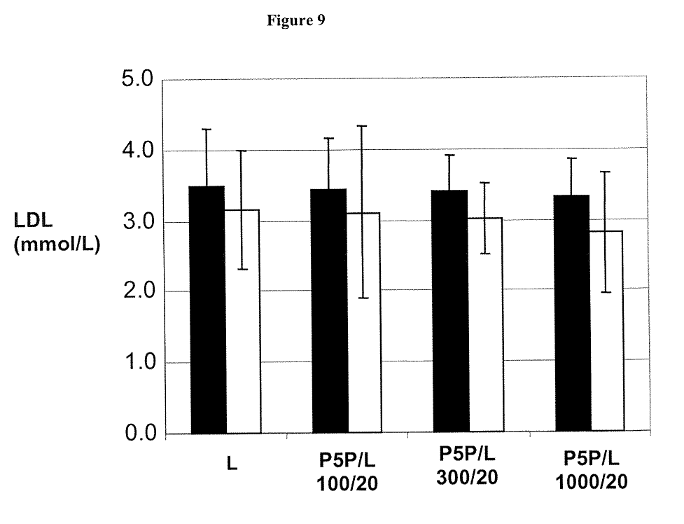

The combination of pyridoxal-5′-phosphate and lisinopril reduced low density lipoprotein (LDL) levels from baseline after sixteen weeks of therapy in patients with a LDL cholesterol level of greater than 2.6 mmol/L at baseline (FIG. 9). In patients receiving lisinopril (20 mg/dose) and placebo, LDL levels were reduced by 0.34 mmol/L (13.3 mg/dL) after 16 weeks of treatment (p=0.164). After sixteen weeks of sequence 1 and sequence 2, administering a 20 mg/dose of lisinopril in combination with 100 mg/dose, 300 mg/dose, or 1000 mg/dose of pyridoxal-5′-phosphate further lowered patients' triglyceride levels from baseline by 0.32 mmol/L (12.5 mg/dL, p=0.205), 0.41 mmol/L (16.0 mg/dL, p=0.045 thereby statistically significant since p<0.05), and 0.53 mmol/L (20.7 mg/dL, p=0.075), respectively.

The data indicate that pyridoxal-5′-phosphate in combination with lisinopril, an ACE inhibitor, further reduced blood pressure and provided better control of glucose and lipid levels than lisinopril alone. Surprisingly, the combination of 300 mg/dose pyridoxal-5′-phosphate and 20 mg/dose lisinopril further reduced blood pressure and provided better glucose control than not only the lower dose pyridoxal-5′-phosphate (100 mg/dose) combined with lisinopril but also the higher dose pyridoxal-5′-phosphate (1000 mg/dose) combined with lisinopril. The enhanced effects of pyridoxal-5′-phosphate appear to be most effective at a middle dose. Although it may be counterintuitive, just administering more of the drug does not provide more relief to high blood pressure and glucose control. These data indicate a more preferred effective dose of pyridoxal-5′-phosphate to be combined with lisinopril, or any ACE inhibitor, is about 300 mg/dose for treating or controlling blood pressure or glucose control in patients with diabetes.

It should be noted that, as used in this specification and the appended claims, the singular forms “a,” an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

1. A method of treating or inhibiting hypertension in a diabetic patient comprising administering a therapeutically effective amount of an angiotensin converting enzyme (ACE) inhibitor and pyridoxal-5′-phosphate, pharmaceutically acceptable acid addition salts thereof, or mixtures thereof, wherein the pyridoxal-5′-phosphate is in a range of about 225 mg to about 500 mg per dose.

2. The method according to claim 1, wherein the ACE inhibitor is selected from a group consisting of benazepril, captopril, cilazapril, enalapril, enalaprilat, fosinopril, lisinopril, moexipril, perindopril, quinapril, ramipril, trandolapril, and a mixture thereof.

3. The method according to claim 1, wherein the ACE inhibitor is lisinopril and the therapeutically effective amount of lisinopril is between 5 and 40 mg per day.

4. The method according to claim 3, wherein the therapeutically effective amount of lisinopril is 20 mg per day.

5. The method according to claim 1, wherein the ACE inhibitor is captopril.

6. The method according to claim 1, wherein the ACE inhibitor is enalapril.

7. The method according to claim 1, wherein the ACE inhibitor is ramipril.

8. The method according to claim 1, wherein the ACE inhibitor is trandolapril.

9. The method according claim 1, wherein the diabetic patient is an insulin dependent diabetic patient.

10. The method according to claim 1, wherein the diabetic patient is a non-insulin dependent diabetic patient.

11. A method of controlling blood glucose or serum glucose in a diabetic patient comprising administering a therapeutically effective amount of an angiotensin converting enzyme (ACE) inhibitor and pyridoxal-5′-phosphate, pharmaceutically acceptable acid addition salts thereof, or mixtures thereof, wherein the pyridoxal-5′-phosphate is in a range of about 225 mg to about 500 mg per dose.

12. The method according to claim 11, wherein the ACE inhibitor is selected from a group consisting of benazepril, captopril, cilazapril, enalapril, enalaprilat, fosinopril, lisinopril, moexipril, perindopril, quinapril, ramipril, trandolapril, and a mixture thereof.

13. The method according to claim 11, wherein the ACE inhibitor is lisinopril and the therapeutically effective amount of lisinopril is between 5 and 40 mg per day.

14. The method according to claim 13, wherein the therapeutically effective amount of lisinopril is 20 mg per day.

15. The method according to claim 11, wherein the ACE inhibitor is captopril.

16. The method according to claim 11, wherein the ACE inhibitor is enalapril.

17. The method according to claim 11, wherein the ACE inhibitor is ramipril.

18. The method according to claim 11, wherein the ACE inhibitor is trandolapril.

19. The method according to claim 11, wherein the diabetic patient is an insulin dependent diabetic patient.

20. The method according to claim 11, wherein the diabetic patient is a non-insulin dependent diabetic patient.
